(19) United States
(12) Patent Application Publication
(10) Pub. No.: US 2016/0113518 A1
(43) Pub. Date: Apr. 28, 2016

(54) MICROCHIP SENSOR FOR CONTINUOUS MONITORING OF REGIONAL BLOOD FLOW

(71) Applicant: THE FEINSTEIN INSTITUTE FOR MEDICAL RESEARCH, Manhasset, NY (US)

(72) Inventors: Raj K. Narayan, Sands Point, NY (US); Chunyan Li, Franklin Square, NY (US)

(73) Assignee: THE FEINSTEIN INSTITUTE FOR MEDICAL RESEARCH, Manhasset, NY (US)

(21) Appl. No.: 14/919,138

(22) Filed: Oct. 21, 2015

Related U.S. Application Data
(60) Provisional application No. 62/068,079, filed on Oct. 24, 2014.

(51) Int. Cl. A61B 5/026

(52) U.S. Cl. CPC A61B 5/026

(57) ABSTRACT

A sensor is provided available for continuous monitoring of regional blood flow in a tissue, including cerebral tissue. Methods of monitoring regional blood flow using the sensor as well as systems and computer readable medium therefor are also provided.

### CROSS-REFERENCE TO RELATED APPLICATIONS

This application claims benefit of U.S. Provisional Application No. 62/068,079, filed Oct. 24, 2014, the contents of which are hereby incorporated by reference.

### STATEMENT OF GOVERNMENT SUPPORT

This invention was made with government support under grant number W81XWH-1-09 awarded by the U.S. Department of Defense. The government has certain rights in the invention.

### BACKGROUND OF THE INVENTION

Throughout this application various patents and other publications are referred to in parenthesis. Full citations for the references may be found at the end of the specification. The disclosures of these patents and publications are hereby incorporated by reference in their entirety into the subject application to more fully describe the art to which the subject invention pertains.

The common mechanism for brain injury-induced neuronal loss is inadequate cerebral blood flow (CBF) for the neurons. During pathological conditions such as traumatic brain injury (TBI) or subarachnoid hemorrhage (SAH), CBF is considered an important upstream monitoring parameter that is indicative of tissue viability (1-3). Hence, monitoring of CBF plays an important role in neurosurgical practice. Continuous monitoring of CBF could provide the opportunity to diagnose and to correct insufficient CBF before deficits in tissue oxygenation and metabolism are recognized. Many techniques are available to assess CBF, such as stable xenon-enhanced computed tomography, single-photon-emission computed tomography, magnetic resonance imaging, positron emission tomography and laser-doppler flometry. However, few of these techniques lend themselves to routine clinical application due to enduring technical drawbacks. Recently, the thermal diffusion flowmetry-based measurement technique which allows the direct and quantitative assessment of regional cerebral perfusion represents a promising monitoring tool in the management of head injured patients (4-5).

The present invention addresses the need for improved devices and methods to assess and/or monitor regional blood flow in a tissue, especially cerebral blood flow.

### SUMMARY OF THE INVENTION

A blood flow meter is provided for monitoring blood flow in a biological tissue comprising (a) a flow sensor comprising a first microelectrode, which flow sensor is capable of being heated by (b) a heater element positioned within 0.5 μm-100 μm of at least a portion of (a), and (c) a temperature sensor comprising a second microelectrode, wherein (a) and (c) are arranged on a flexible substrate and wherein (b) is positioned relative to (c) such that when (a) is heated by (b) to a stable target temperature of 0.5° C. to 3° C. above the temperature of the biological tissue in which the blood flow meter is situated, (c) is not within the thermal influence field generated by (b).

Also provided is a blood flow meter for monitoring blood flow in a biological tissue, comprising (i) a flexible substrate; (ii) a flow sensor (x) comprising a first microelectrode, which flow sensor is capable of being heated by (iii) a heater element (y) positioned within 0.5 μm-100 μm of at least a portion of (x), wherein (x) is positioned on a first flexible substrate layer of a flexible substrate and (y) is positioned on a second flexible substrate layer of the flexible substrate, and wherein (x) and (y) are separated from each other by at least an intervening silicon nitride layer, or other humidity diffusion layer such as, in non-limiting examples, aluminum oxide, boron nitride.

In another aspect of the invention, an array is provided of two of the blood flow meters described herein wherein the first blood flow meter and second blood flow meters are spatially separated on the same flexible substrate layers such that the first microelectrode of the first blood flow meter is outside the thermal influence field of the heater of the second blood flow meter and the first microelectrode of the second blood flow meter is outside the thermal influence field of the heater of the first blood flow meter.

A method is also provided for determining a blood flow in a tissue of subject comprising measuring the blood flow with a blood flow meter described herein situated in the tissue of the subject.

Also provided is a system for monitoring a blood flow in a tissue of a subject, comprising:

one or more data processing apparatus coupled to a blood flow meter as described herein; and
a computer-readable medium coupled to the one or more data processing apparatus having instructions stored thereon which, when executed by the one or more data processing apparatus, cause the one or more data processing apparatus to perform a method as described herein so as to monitor the blood flow in a tissue of a subject.

A non-transitory computer-readable medium is provided comprising instructions stored thereon which, when executed by a data processing apparatus, causes the data processing apparatus to perform a method as described herein so as to monitor blood flow in a subject.

Additional objects of the invention will be apparent from the description which follows.

### BRIEF DESCRIPTION OF THE DRAWINGS

FIG. 1: Schematic of the improved cerebral blood flow sensor.

FIG. 2: Block diagram of the improved circuit design for the flow sensor.

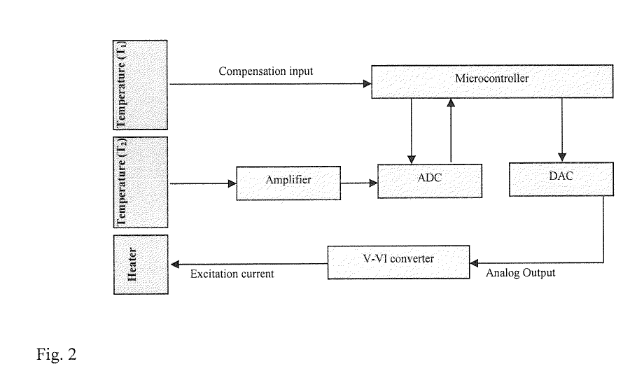

FIG. 3(A)-3(C): In vitro accuracy/stability test results: (A) long-term stability test and (B) low frequency noise (HPF: 0.001 Hz) test. The 3-element approach where the heater is on the top and temperature sensor is underneath achieves highest stability and accuracy. The best results are achieved when the heater is positioned in between the temperature sensor (under the heater) and organ tissue/blood flow.

FIG. 4: A 2-element flow sensor.

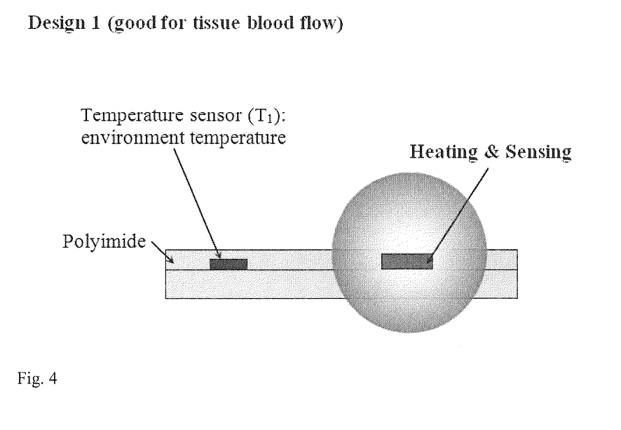

FIG. 5: With the three-element design a much higher accuracy and stability can be achieved (especially if there is a large flow change and/or temperature change). Also a faster response time is effected. T1 and T2 can be a thermocouple, a thermistor or thin film resistive temperature detector (RTD). Also a high stability is achieved both in continuous and periodic heating cases.

FIG. 6: Using rapid heating and cooling, no additional temperature sensor is necessary to do an environmental temperature compensation. By rapid heating and cooling, the thermal profile from the heater is reduced, hence reducing the risk of a heating pulse being transported to a remote site, which is critical, for example, in the area of cardiovascular monitoring.

FIG. 7: By using multiple arrays continuous monitoring can be effected, which means that the flow can be monitored from one sensor of the blood flow meter even during the cooling period or calibration period of one (or more) other flow sensor(s) of the blood flow meter.

FIG. 8: Active cooling using thermoelectric thin-film dramatically reduces cooling time permitting an increase the sampling rate. In addition, the heating temperature range can be increased. For example, if the heater itself has 3° C. lower temperature than ambient, in order to heat the flow sensor 2° C. higher, one can heat 5° C., which means higher sensitivity and a wider detection range can be achieved.

FIG. 9: An embodiment where a 3-element cerebral blood flow senor was realized by 2-layer microfabrication, where the heater is on the top layer and the temperature sensor measuring heater temperature is on the bottom layer.

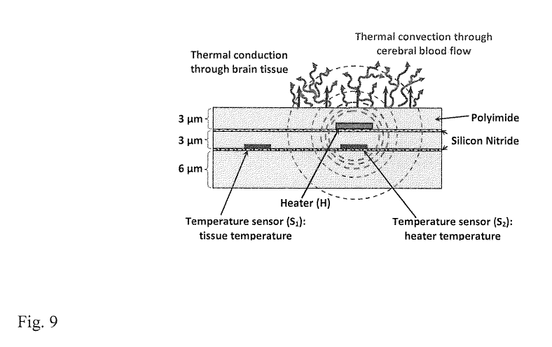

FIG. 10: An embodiment where a 3-element cerebral blood flow senor was realized by single-layer microfabrication, where the heater and temperature sensor measuring heater temperature are on the same layer. The patterns for heater and temperature sensor were interdigitated to have uniform heating and accurate temperature sensing.

FIG. 11: An air gap added under the heater to prevent thermal conduction. This pattern has the advantage of being used as a “smart skin” that can detect the surface blood flow for any organ.

### DETAILED DESCRIPTION OF THE INVENTION

A blood flow meter is provided for monitoring blood flow in a biological tissue comprising (a) a flow sensor comprising a first microelectrode, which flow sensor is capable of being heated by (b) a heater element positioned within 0.5 μm-100 μm of at least a portion of (a), and (c) a temperature sensor comprising a second microelectrode, wherein (a) and (c) are arranged on a flexible substrate and wherein (b) is positioned relative to (c) such that when (a) is heated by (b) to a stable target temperature of 0.5° C. to 3° C. above the temperature of the biological tissue in which the blood flow meter is situated, (c) is not within the thermal influence field generated by (b).

In an embodiment, the flexible substrate comprises at least 5 (five) layers.

In an embodiment, at least two layers of the flexible substrate comprise a flexible polymer. In an embodiment, at least three layers of the flexible substrate comprise a flexible polymer. In an embodiment, at least two layers of the flexible substrate comprise silicon nitride. In an embodiment, an uppermost layer of the flexible substrate comprises silicon nitride and wherein a lowermost layer of the flexible substrate comprises silicon nitride.

In an embodiment, the flexible substrate comprises at least 6 layers comprising, from top to bottom, with the bottom layer being exposed to the biological tissue or a blood vessel where blood flow is to be measured: a first silicon nitride layer adjacent to a first polyimide layer adjacent to a second silicon nitride layer adjacent to a second polyimide layer adjacent to a third polyimide layer adjacent to a third silicon nitride layer.

In an embodiment, each layer is substantially continuous with its one or more adjacent layer(s).

In an embodiment, (a) and (c) are separated from (b) by at least an intervening silicon nitride layer.

In an embodiment, (a) and (c) are arranged on the same layer of the flexible substrate.

In an embodiment, (b) is oriented relative to (a) such that (a) is further away from the blood flow that is being measured than (b) is.

In an embodiment, (b) is oriented relative to (a) such that (b) is further away from the blood flow that is being measured than (a) is.

In an embodiment, the blood flow is measured in a vessel or in a tissue adjacent to the bottom sodium nitride layer.

In an embodiment, the first microelectrode is positioned atop the third polyimide layer and within the second polyimide layer. In an embodiment, the second microelectrode is positioned atop the third polyimide layer and within the second polyimide layer. In an embodiment, the heater element is positioned atop the second silicon nitride layer and within the first polyimide layer.

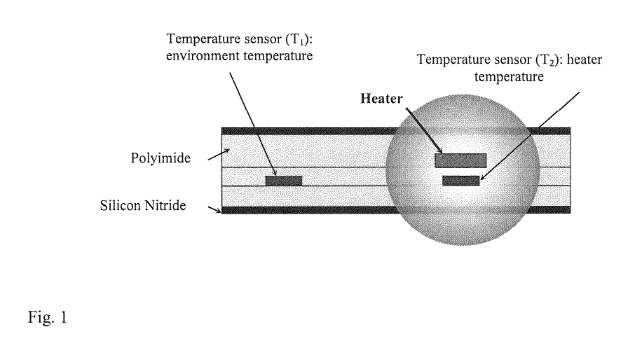

In an embodiment, a configuration as shown in FIG. 1 or Design 2.

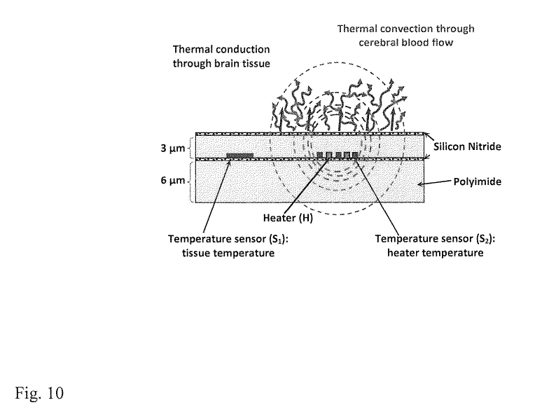

In an embodiment, the blood flow meter has a configuration as shown in FIG. 10.

In an embodiment of the blood flow meter, (b) and (a) are arranged on the same layer of the flexible substrate and are interdigitated.

In an embodiment, the blood flow meter has an air gap immediately adjacent to a polyimide layer underlying the interdigitated (a) and (b).

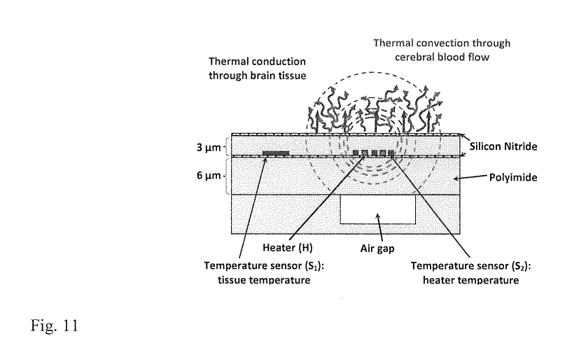

In an embodiment, the blood flow meter has a configuration as shown in FIG. 11.

Also provided is a blood flow meter for monitoring blood flow in a biological tissue, comprising (i) a flexible substrate; (ii) a flow sensor (x) comprising a first microelectrode, which flow sensor is capable of being heated by (iii) a heater element (y) positioned within 0.5 μm-100 μm of at least a portion of (x), wherein (x) is positioned on a first flexible substrate layer of a flexible substrate and (y) is positioned on a second flexible substrate layer of the flexible substrate, and wherein (x) and (y) are separated from each other by at least an intervening silicon nitride layer.

In an embodiment, the flexible substrate comprises at least 6 layers.

In an embodiment, at least two layers of the flexible substrate comprise a flexible polymer. In an embodiment, at least three layers of the flexible substrate comprise a flexible polymer. In an embodiment, at least two layers of the flexible substrate comprise silicon nitride. In an embodiment, at least three layers of the flexible substrate comprise silicon nitride.

In an embodiment, the flexible substrate comprises at least 6 layers comprising, from top to bottom with the bottom layer being exposed to the biological tissue, or a blood vessel therein, where blood flow is to be measured, a first silicon nitride layer adjacent to a first polyimide layer adjacent to a second silicon nitride layer adjacent to a second polyimide layer adjacent to a third polyimide layer adjacent to a third silicon nitride layer.

In an embodiment, each layer is substantially continuous with its one or more adjacent layer(s). In an embodiment, (x) is separated from (y) by at least two intervening silicon nitride layers. In an embodiment, (x) is oriented relative to (y) such that (y) is further away from the blood flow that is being measured than (x) is.

In an embodiment, the blood flow is measured in a vessel or in a tissue adjacent to the bottom sodium nitride layer.

In an embodiment, the first microelectrode is positioned atop the third polyimide layer and within the second polyimide layer.

In an embodiment, the different layers are, independently, from 1 μm to 100 μm thick. In an embodiment, the different layers are, independently, from 3 μm to 20 μm thick.

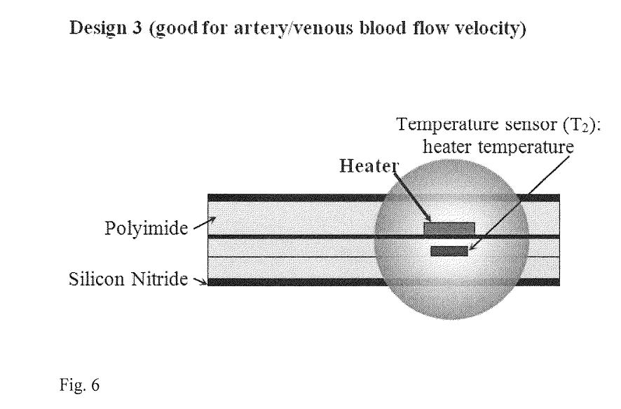

In an embodiment, the blood flow meter has a configuration as shown in FIG. 6 or Design 3.

In an embodiment, the blood flow meter further comprises a further silicon nitride layer positioned underneath the first microelectrode, and a thermoelectric film adjacent to the further silicon nitride layer, at least a portion of which thermoelectric film is positioned directly under the portion of the further silicon nitride layer that is directly underneath the first microelectrode.

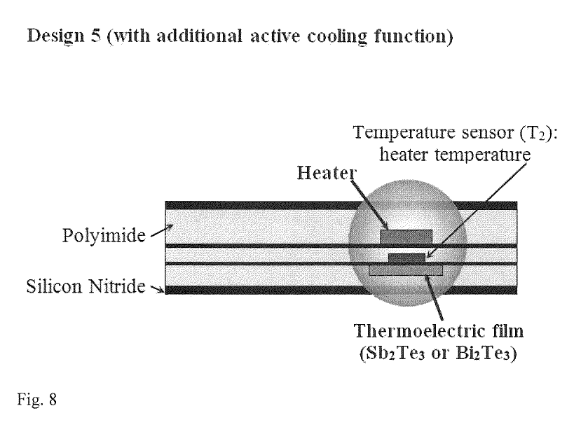

In an embodiment, the blood flow meter has a configuration as shown in FIG. 8 or Design 5.

Also provided is an array of any two, or more, of the blood flow meters described herein, wherein the first blood flow meter and second blood flow meters are spatially separated on the same flexible substrate layers such that the first microelectrode of the first blood flow meter is outside the thermal influence field of the heater of the second blood flow meter and the first microelectrode of the second blood flow meter is outside the thermal influence field of the heater of the first blood flow meter. In an embodiment, the two blood flow meters are of the same type.

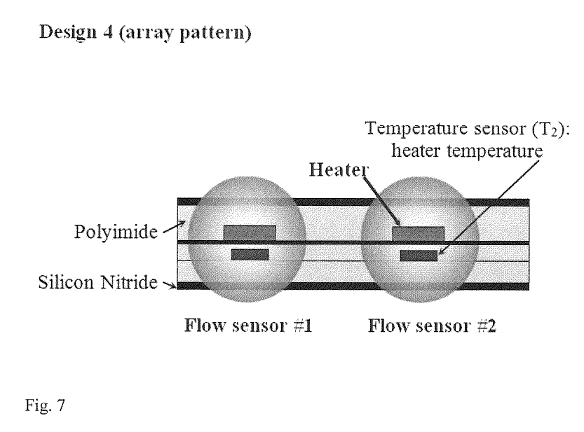

In an embodiment, the array has a configuration as shown in FIG. 7 or Design 4.

In an embodiment, the flow sensor comprising a first microelectrode comprises a 4-wire configuration. In an embodiment, the flow sensor is a constant-temperature flow sensor.

In an embodiment of any of the blood flow meters described herein, the temperature sensor quantitates the temperature of the medium in which the sensor for monitoring blood flow is situated, and wherein the sensor for monitoring blood flow is a component of an electrical circuit such that the output of the flow sensor is corrected for changes in temperature of the medium by the output of the temperature sensor.

In an embodiment of any of the blood flow meters described herein, the electrical circuit comprises an interface circuit. In an embodiment of any of the blood flow meters described herein, the medium comprises the biological tissue.

In an embodiment of any of the blood flow meters described herein, the microelectrode is continuous with an electrically-conducting wire, each of which wires are electroplated with a material to reduce lead resistances thereof In an embodiment, the electrically-conducting wires are electroplated with copper. In an embodiment, the microelectrodes comprise gold. In an embodiment, the microelectrodes comprise gold.

In an embodiment, the electrically-conducting wires further comprise a flexible insulator coating. In an embodiment, the flexible insulator comprises poly(4,4′-oxydiphenylene-pyromellitimide). In an embodiment, the electrically-conducting wires further comprise one or more poly(p-xylylene) polymers. In an embodiment, (a) is capable of being heated to 0.1° C. to 3.5° C. above the temperature of the tissue in which the sensor is situated for a continuous time period of up to 20 seconds.

In an embodiment of any of the blood flow meters described herein, the sensor is integrated on an assembly further comprising one or more of a pressure sensor, a pH sensor, a glucose sensor, a microdialysis probe, an oxygen sensor, a lactate sensor, a pyruvate sensor, a glutamate sensor, and a carbon dioxide sensor. In an embodiment of any of the blood flow meters described herein, the blood flow meter is fabricated as a flexible spirally-rolled polymer microtube. In an embodiment of any of the blood flow meters described herein, the blood flow meter is fabricated as a catheter for blood vessels, or is fabricated to be placed inside a catheter for blood vessels.

In an embodiment of any of the blood flow meters described herein, the blood flow meter is operationally connected to a control device which controls the input current and/or voltage into the temperature sensor and which controls the input current and/or voltage into the flow sensor.

In an embodiment of any of the blood flow meters described herein, the blood flow meter is operationally connected to one or more data processors which receive and process the output of the flow sensor and/or temperature sensor. In an embodiment of any of the blood flow meters described herein, one or more data processors compensate the output of the flow sensor for changes in the temperature of the medium as determined from the output of the temperature sensor. In an embodiment of any of the blood flow meters described herein, one or more data processors further compensate the output of the flow sensor for changes in the thermal conductivity of the medium as determined from the peak output of the flow sensor during a period in which (a) is heated. In an embodiment, the thermal conductivity of the medium is determined from sampling the peak initial current required to heat (a) to a stable target temperature of 1° C. to 3° C. above the temperature of the tissue in which the sensor is situated. In an embodiment, the peak initial current is sampled when the flow sensor temperature is 0.05° C. to 0.3° C. above the stable target temperature. In an embodiment, the peak initial current is sampled for 25 mS to 200 mS. In an embodiment, the peak initial current is sampled for 75 mS to 125 mS. In an embodiment, the thermal conductivity of the medium is determined from the square of average of the sampling of two peak initial currents.

In an embodiment of any of the blood flow meters described herein, the blood flow rate is determined from the output of the flow sensor, compensated for thermal conductivity of the medium, and compensated for changes in temperature of the medium. In an embodiment of any of the blood flow meters described herein, the first microelectrode comprises a thermoresistive microelectrode and/or wherein the second microelectrode comprises a thermoresistive microelectrode. In an embodiment of any of the blood flow meters described herein, the sensor is capable of being heated to a stable target temperature of 1° C. to 3° C. above the temperature of the tissue and subsequently cooling to the temperature of the tissue within a total period of 5-10 seconds.

In an embodiment of any of the blood flow meters described herein, voltage output of the blood flow meter is proportional to the blood flow rate.

Also provided is method for determining a blood flow in a tissue of subject comprising measuring the blood flow with any of the blood flow meters described herein, or array thereof, situated in the tissue of the subject.

In an embodiment, the method comprises determining a baseline tissue temperature by causing a non-heating current to be applied to the sensor for monitoring blood flow and measuring the output of the sensor. In an embodiment, the non-heating current is applied for between 0.1 and 20 seconds.

In an embodiment, the method comprises effecting the temperature of the flow sensor to stably be 1° C. to 3° C. above the temperature of the tissue in which the blood flow meter is situated by causing a heating current to be applied to the sensor.

In an embodiment, the method comprises effecting the temperature of the flow sensor to stably be 1.9 to 2.1° C. above the temperature of the tissue in which the sensor is situated by causing a heating current to be applied to the sensor.

In an embodiment, the heating current is applied for between 0.1 and 20 seconds.

In an embodiment, the method comprises measuring the output of the flow sensor during the last 5 seconds of the period during which the heating current is applied.

In an embodiment, the method comprises measuring the output of the flow sensor during the last quarter of the period during which the heating current is applied.

In an embodiment, the method further comprises determining the thermal conductivity of the cerebral tissue by sampling the peak initial current required to heat the flow sensor to stably be 1° C. to 3° C. above the temperature of the cerebral tissue.

In an embodiment, the tissue blood flow is measured at a plurality of discrete time points using the sensor for monitoring blood flow. In an embodiment, the sensor for monitoring blood flow is permitted to cool after termination of a first heating current and before initiation of a second heating current being applied. In an embodiment, the period during which the sensor for monitoring blood flow is permitted to cool is 2 to 20 seconds.

In an embodiment, the method comprises determining a baseline tissue temperature by causing a non-heating current to be applied to the sensor for monitoring blood flow and measuring the output of the sensor during the period during which the sensor for monitoring blood flow is permitted to cool.

In an embodiment, the tissue is cerebral tissue.

In an embodiment, the method comprises determining the baseline cerebral tissue temperature during the terminal portion of the time period during which the sensor for monitoring blood flow is permitted to cool and immediately before the a heating current is applied.

In an embodiment, the method further comprises placing the blood flow meter in a cerebral blood vessel of the subject.

In an embodiment, the total of the heating period and the subsequent cooling period is 5-10 seconds.

In an embodiment, the subject has suffered a brain injury or is undergoing a surgery or a therapeutic intervention upon the brain.

As used herein a “blood flow meter” is a device for measuring, quantitating and/or monitoring blood flow.

As used herein a “sensor” is a device that measures a parameter and converts it into a signal which can be read by an observer or by an instrument. In the case of a temperature sensor, temperature is being measured. In the case of a liquid flow sensor, flow is calculated from another parameter measured, such as temperature. The device may be a microelectrode-based device, for example a thermoresistive microelectrode-based device. The output is an electrical output (e.g. a signal) which is related, for example proportional, to the parameter being determined, such as temperature.

As used herein a “biological tissue” is any tissue with a blood supply in an animal, or which has been removed from an animal. As such, biological tissue includes, in non-limiting examples, a tissue in situ and a tissue which has been removed from, e.g. a human, for transplant purposes. In an embodiment, the biological tissue is in vivo.

“Adjacent to,” as used herein, means immediately next to, spatially. In an embodiment, adjacent to, with regard to layers, encompasses layers at least partially adhered to one another. In an embodiment, adjacent to, with regard to layers, includes layers immediately next to one another but not adhered to one another.

As used herein “substantially continuous” with regard to two entities each having sides, means the two entities touching each over a majority of at least one side of each.

In an embodiment of the invention, the blood flow meter can measure, quantitate and/or monitor liquid flow, such as blood flow, but not gas flow.

In an embodiment, the variously described layers are continuous for the majority of the length of the flexible substrate. In an embodiment, the variously described layers are continuous for the whole length of the flexible substrate.

In an embodiment, the variously described layers are continuous for the majority of the width of the flexible substrate. In an embodiment, the variously described layers are continuous for the whole width of the flexible substrate.

The thermal influence field generated by, for example, a heater, and as represented schematically as the circle in FIG. 1, is the area around the heater within which an actual and measurable increase in temperature occurs upon a relevant temperature sensor (e.g., T₂ in FIG. 1) being heated to a predetermined stable target temperature. The temperature sensor outside the thermal influence field (e.g., T₁ in FIG. 1), is not in the thermal influence field in that no measurable increase in temperature of T₁ occurs upon the temperature sensor T₂ in FIG. 1 being heated to the predetermined stable target temperature. A preferred range of physical distance between T₂ (or equivalent) and the heater is from 3 μm to 20 μm, inclusive. A preferred range of physical distance between T₁ (or equivalent) and the heater is 1-6 mm, inclusive.

The blood flow meter is preferably small enough in size to be inserted into blood vessels. In an embodiment the sensor for monitoring blood flow is 10 μm-25 μm thick. In a preferred embodiment the device comprising the sensor for insertion into a tissue is no more than 20 μm thick. In a most preferred embodiment the device comprising the sensor for insertion into a tissue is no more than 15 μm thick. In an embodiment the device comprising the sensor for insertion into a tissue is 10 μm-25 μm thick.

The flexibility of the sensor reduces tissue damage when the sensor is placed and aids insertion into blood vessels. The sensor can be fabricated using any means known in the art, including, in a non-limiting example, spirally rolling technology. For example, the sensor can be fabricated as a flexible spirally-rolled polymer microtube, the fabrication of which is described in U.S. Patent Application Publication No. 2009/0297574 A1, the contents of which are hereby incorporated by reference. In an embodiment, the sensor for monitoring blood flow is fabricated as a catheter for blood vessels, or is fabricated to be placed inside a catheter for blood vessels.

The blood flow meter can be part of, or integrated into, an assembly further comprising one or more of a pressure sensor, a pH sensor, a glucose sensor, a microdialysis probe, an oxygen sensor, a lactate sensor, a pyruvate sensor, a glutamate sensor, and/or a carbon dioxide sensor.

In an embodiment, the blood flow meter can be operated to ratiometrically measure the resistance of each the sensors/microelectrodes of which it is comprised. This results in a more precise measurement than bridge-type thermal diffusion flow sensors. In an embodiment the flow sensor comprising a first microelectrode comprises a 4-wire configuration. This advantageously eliminates lead wire effect.

In an embodiment the temperature sensor comprising a first microelectrode comprises a 4-wire configuration

In an embodiment, the temperature sensor quantitates the temperature of the medium in which blood flow meter is situated, and is a component of an electrical circuit such that the output is corrected for changes in temperature of the medium by the output of the temperature sensor. In a preferred embodiment, the medium comprises the biological tissue. Accordingly, in an embodiment, the blood flow meter is a constant-temperature flow sensor, which compensates for medium baseline temperature shifts and thus provides improved accuracy of measurement. In a preferred embodiment, the electrical circuit comprises an interface circuit.

The microelectrodes are each continuous with electrically-conducting wires which are preferably electroplated with a material to reduce lead resistances thereof. Suitable electroplating material for the wires includes copper. In an embodiment, the wires are coated with copper 1 to 4 μm thick. In a preferred embodiment, the wires are coated with copper 1.5 to 2.5 μm thick. In a most preferred embodiment, the wires are coated with copper 2 μm thick. The microelectrode can be can be fabricated by depositing one or more conducting materials, such as metals, on a suitable base such as an insulator. In a preferred embodiment the microelectrodes comprise gold. In a preferred embodiment the microelectrodes further comprise titanium. In an embodiment the gold is deposited in a layer 800-1600 Å thick, for example, on a flexible insulator. Suitable insulators include, in a non limiting example, a poly(4,4′-oxydiphenylene-pyromellitimide). In a preferred embodiment, the gold is deposited in a layer 1000-1400 Å thick. In a most preferred embodiment, the gold is deposited in a layer 1200 Å thick. In a preferred embodiment, the titanium is deposited on top of the gold in a layer 80-200 Å thick. In a preferred embodiment, the titanium is deposited in a layer 120-180 Å thick. In a most preferred embodiment, the titanium is deposited in a layer 150 Å thick. In an embodiment, the insulator is 5-15 μm thick. In a preferred embodiment, the insulator is 6-10 μm thick. In a most preferred embodiment, the insulator is 7.5 μm thick. The microelectrodes may be completed using thin film lithography and etching processes known in the art. The components may further be coated with one or more poly(p-xylylene) polymers. In an embodiment, the poly(p-xylylene) polymer is 2-10 μm thick. In a preferred embodiment, the poly(p-xylylene) polymer is 4-7 μm thick. In a most preferred embodiment, the poly(p-xylylene) polymer is 5 μm thick.

As used herein a “flexible substrate” is any suitable substrate which does not evoke an inflammatory and/or immune response when in situ in most human subjects, i.e. is inert. While it is not possible to exclude any one individual from having an inflammatory and/or immune response to a foreign material, many materials are accepted to be generally inert. Such materials which are flexible and can be used as flexible substrates are known in the art. Preferably, the flexible substrate comprises one or more flexible polymer layers. Examples of one class of flexible polymers that can be used in the present invention are polyimides. Poly(p-xylylene), and polyvinylidene fluoride trifluoroethylene (PDVF-TrFE), poly-lactic-co-glycolic acid (PLGA), polyethylene, and polydimethylsiloxane (PDMS) may also be used.

Polyimides (PI) have been used successfully as a substrate and insulation material for implants. However, high water absorption as well as high oxygen permeation of PI limit the performance of those microsensors using PI as a substrate. Herein, a permeability-reducing layer(s) is/are preferably incorporated into the flexible substrate. In a preferred embodiment, thin silicon nitride film (e.g., 40 nm˜100 nm) is sputtered on a flexible PI substrate to reduce water or gas permeability and so improve sensor performance.

In an embodiment, an intervening silicon nitride layer as referred to herein is the second silicon nitride layer.

In an embodiment the heater element is a micro-heater comprising patterned Au or Pt film (e.g., 1200 nm thick, 20 μm wide). Temperature sensors as set forth in the designs in the figures T₁ and T₂ are, in embodiments, Au or Pt RTD (Resistance Temperature Detector) or thin film thermistor or thermocouple.

In an embodiment, the blood flow meter is operationally connected to a control device which controls the input current and/or voltage into the temperature sensor and which controls the input current and/or voltage into the flow sensor. In a most preferred embodiment, the sensor is operationally connected to one or more data processors which receive and process the output of the flow sensor and/or temperature sensor. The data processors can, and preferably do, compensate the output of the flow sensor for changes in the temperature of the medium (e.g. changes in the local temperature of the brain) as determined from the output of the temperature sensor.

In an embodiment, the blood flow meter can also correct for the thermal conductivity of the medium in which the device is placed. In a preferred embodiment, the one or more data processors further compensates the output of the flow sensor for changes in the thermal conductivity of the medium as determined from the peak output of the flow sensor during a period in which (a) is heated.

The blood flow rate can be determined from the output of the flow sensor compensated for thermal conductivity of the medium and compensated for changes in temperature of the medium. In preferred embodiments, the linear coefficient of R² for the correlation of output voltage to blood flow is in excess of 0.95. In a most preferred embodiment, the linear coefficient of R² for the correlation is in excess of 0.99.

The blood flow can be measured using the described sensor in any biological tissue. In an embodiment, the tissue is a cerebral tissue and the blood flow is a cerebral blood flow.

In another aspect of the invention methods are provided for determining a blood flow in a tissue of subject comprising measuring the blood flow using the a blood flow meter as described herein, situated in the tissue of the subject.

In an embodiment, a baseline tissue temperature is determined comprising causing a non-heating current to be applied to the sensor for monitoring blood flow and measuring the output of the sensor. In an embodiment, the non-heating current is applied for between 0.1 and 20 seconds. In a preferred embodiment, the non-heating current is applied for between 2 and 8 seconds. In a more preferred embodiment, the non-heating current is applied for between 3 and 6 seconds.

In embodiments, the method also comprises effecting a temperature of the flow sensor to stably be 0.5° C. to 3° C. above the temperature of the tissue, for example cerebral tissue, in which the sensor is situated by causing a heating current to be applied to the sensor. In a preferred embodiment, a stable temperature of 1° C. to 2.5° C. above the temperature of the tissue is effected. In a most preferred embodiment, a stable temperature of 2° C. above the temperature of the tissue is effected. In an embodiment, to effect a temperature of the flow sensor to be above the temperature of the tissue, the heating current is applied for between 0.1 and 20 seconds. In a preferred embodiment, the heating current is applied for between 2 and 8 seconds. In a most preferred embodiment, the heating current is applied for 3 seconds, 4 seconds or for a time period in between 3 and 4 seconds.

The output of the flow sensor is preferably measured during the posterior portion of the period during which the heating current is applied. In an embodiment, measuring the output of the flow sensor is effected during the last 0.1 to 8 seconds of the period during which the heating current is applied. In an embodiment, measuring the output of the flow sensor is effected during the last 0.1 to 6 seconds of the period during which the heating current is applied. In an embodiment, measuring the output of the flow sensor is effected during the last 0.1 to 5 seconds of the period during which the heating current is applied. In an embodiment, measuring the output of the flow sensor is effected during the last 0.1 to 4 seconds of the period during which the heating current is applied. In a preferred embodiment, measuring the output of the flow sensor is effected during the last 0.1 to 3 seconds of the period during which the heating current is applied. In a most preferred embodiment, measuring the output of the flow sensor is effected during the last 0.5 to 2 seconds of the period during which the heating current is applied. In an embodiment, measuring the output of the flow sensor is effected during the last 1 second of the period during which the heating current is applied. In an embodiment, the output of the flow sensor during the last third, last quarter or last fifth of the period during which the heating current is applied.

In embodiments, the device and/or methods described herein are used in the early detection of vasospasm and other conditions of compromised perfusion. In an embodiment of the methods, the tissue is cerebral tissue. In a preferred embodiment, the device and/or methods described herein are used in monitoring cerebral blood flow when managing secondary injury in traumatic brain injury subjects. In another preferred embodiment, the device and/or methods described herein are used in monitoring cerebral blood flow during neurosurgical applications, for example during neurosurgery upon the subject and/or in the recovery period after neurosurgery upon the subject. In an embodiment of the methods, the tissue is a cardiovascular tissue. In an embodiment, the device and/or methods described herein are used in monitoring a cardiovascular tissue blood flow, for example during cardiac surgery upon the subject and/or in the recovery period after cardiac surgery upon the subject. In a preferred embodiment of the methods, the subject is a human.

In embodiments, the device and/or methods described herein are used to monitor blood flow in a non-cerebral tissue. In embodiments, the device and/or methods described herein are used to monitor blood flow in a tissue of an organ of a mammal In a preferred embodiment, the tissue, or organ itself, is a tissue about to be transplanted or being transplanted or having been transplanted. The methods and devices described herein can be applied mutatis mutandis to measure fluid flow in a tissue, as opposed to blood flow. In an embodiment, the fluid is an organ viability-preserving fluid. In an embodiment of the organ is a lung, kidney, liver, pancreas, intestine, thymus or heart.

The reading can be corrected for by compensating for the thermal conductivity of the tissue. In an embodiment, the method comprises determining the thermal conductivity of the cerebral tissue by sampling the peak initial current required to heat the flow sensor to stably be 1° C. to 3° C. above the temperature of the cerebral tissue. In a preferred embodiment, the method comprises determining the thermal conductivity of the cerebral tissue by sampling the peak initial current required to heat the flow sensor to stably be 1.5° C. to 2.5° C. above the temperature of the cerebral tissue. In a most preferred embodiment, the method comprises determining the thermal conductivity of the cerebral tissue by sampling the peak initial current required to heat the flow sensor to stably be 2° C. above the temperature of the cerebral tissue.

The method can be used to continuously or discontinuously monitor blood flow as desired. In an embodiment, the tissue blood flow measured at a plurality of discrete time points using the sensor for monitoring blood flow.

Preferably, the sensor for monitoring blood flow is permitted to cool after termination of a first heating current and initiation of a second or subsequent heating current being applied. In an embodiment, the period during which the sensor for monitoring blood flow is permitted to cool is 1 to 20 seconds. In a preferred embodiment, the period during which the sensor for monitoring blood flow is permitted to cool is 2 to 10 seconds. In a most preferred embodiment, the period during which the sensor for monitoring blood flow is permitted to cool is 2 to 5 seconds. In an embodiment, active cooling is effected. In an embodiment, passive cooling is employed. An active cooling function, for example as shown in Design 5 (FIG. 8), can dramatically reduce the cooling time and allow increased sampling rate. This can be effected using a thermoelectric thin-film (e.g., Sb₂Te₃ or Bi₂Te₃). This also provides the option of increasing the heating temperature. For example, if the heater itself has 3° C. lower temperature than ambient, in order to heat the flow sensor 2° C. higher, one can heat 5° C., which means one can achieve higher sensitivity and a wider detection range not possible with previous devices.

A baseline cerebral tissue temperature can be determined by causing a non-heating current to be applied to the sensor for monitoring blood flow and measuring the output of the sensor during the period during which the sensor for monitoring blood flow is permitted to cool. This permits monitoring of the baseline temperature so as to re-calibrate the sensor for any changes in baseline temperature. In an embodiment, the method comprises determining baseline cerebral tissue temperature during the terminal portion of the time period during which the sensor for monitoring blood flow is permitted to cool and immediately before the a heating current is applied.

In an embodiment of the sensor, the sensor is constructed such that the sensor is capable of being heated to a stable target temperature of 1° C. to 3° C. above the temperature of the tissue and subsequently cooling to the temperature of the tissue within a total period of 5-10 seconds. In an embodiment, the sensor is constructed such that the sensor is capable of being heated to a stable target temperature of 1.9° C. to 2.1° C. above the temperature of the tissue and subsequently cooling to the temperature of the tissue within a total period of 5-10 seconds.

In an embodiment of the blood flow meter, the sensor is constructed with a thermoelectric film adjacent to the microelectrode capable of being heated such that the sensor is capable of being heated to a stable target temperature of 1° C. to 3° C. above the temperature of the tissue and subsequently cooling to the temperature of the tissue within a total period of 0.1-4.9 seconds. In an embodiment, the blood flow meter is constructed such that the sensor is capable of being heated to a stable target temperature of 1.9° C. to 2.1° C. above the temperature of the tissue and subsequently cooling to the temperature of the tissue within a total period of 0.1-4.9 seconds.

In an embodiment of the methods, the blood flow meter is constructed with a thermoelectric film adjacent to the microelectrode capable of being heated such that the sensor is capable of being heated to a stable target temperature of 1° C. to 3° C. above the temperature of the tissue and subsequently cooling to the temperature of the tissue within a total period of 0.1-4.9 seconds. In an embodiment, the blood flow meter is constructed such that the sensor is capable of being heated to a stable target temperature of 1.9° C. to 2.1° C. above the temperature of the tissue and subsequently cooling to the temperature of the tissue within a total period of 0.1-4.9 seconds.

In an embodiment, the blood flow meter has a sensitivity of better than 0.5 mV/ml/100 g/min in the range of 0 to 200 ml/100 g/min. In an embodiment, the blood flow meter has a sensitivity of better than 0.54 mV/ml/100 g/min in the range of 0 to 200 ml/100 g/min. In an embodiment, the blood flow meter has an accuracy of equal to or better than 3 ml/100 g/min in vivo in a mammalian cerebral tissue.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions encoded on a computer readable medium for execution by, or to control the operation of, data processing apparatus. The computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

Also provided is a system for monitoring a blood flow in a tissue of a subject, comprising:

one or more data processing apparatus coupled to a blood flow meter as described herein; and
a computer-readable medium coupled to the one or more data processing apparatus having instructions stored thereon which, when executed by the one or more data processing apparatus, cause the one or more data processing apparatus to perform a method as described herein so as to monitor the blood flow in a tissue of a subject.

Also provided is a non-transitory computer-readable medium comprising instructions stored thereon which, when executed by a data processing apparatus, causes the data processing apparatus to perform a method as described herein so as to monitor blood flow in a subject.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The methods, or portions thereof, processes and logic flows described in this specification can be performed by one or more programmable processors executing one or more computer programs to perform functions by operating on input data and generating output. The methods, or portions thereof, processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube), or LED (light emitting diode), or LCD/LED, or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

In embodiments, the methods as described herein when referring to cerebral blood flow or a cardiovascular blood flow can each be applied as stated in concert with, or simultaneously/contemporaneously with, an organ activity imaging/quantification method such as PET and MRI methods (e.g. fMRI of brain activity), SPECT and CT. In embodiments the methods further comprise administering to the subject one or more agents, e.g. radionuclides, necessary to perform the organ activity imaging/quantification. In an embodiment, any two or more of the brain activity imaging/quantification methods can be used together to provide the detail on which the pattern of organ activity is identified. PET images demonstrate the metabolic activity chemistry of an organ, such as the brain. A radiopharmaceutical, such as fluorodeoxyglucose, which includes both sugar and a radionuclide, is injected into the subject, and its emissions are measured by a PET scanner. The PET system detects pairs of gamma rays emitted indirectly by the positron-emitting radionuclide (tracer), which is introduced into the body on a biologically active molecule. Radiopharmaceuticals such as fluorodeoxyglucose as the concentrations imaged can be used as indication of the metabolic activity at that point. Magnetic resonance imaging (MRI) makes use of the property of nuclear magnetic resonance (NMR) to image nuclei of atoms inside the body, e.g. the brain. Strong magnetic field gradients cause nuclei at different locations to rotate at different speeds. 3-D spatial information can be obtained by providing gradients in each direction. In the embodiment of functional MRI (fMRI), the scan is used to measure the hemodynamic response related to neural activity in the brain.

In an embodiment of the methods, the blood flow is measured in an arterial vessel. In another embodiment, the blood flow is measured in a venous vessel.

In a preferred embodiment of the methods and devices herein, the tissue in which the blood flow is measured is a tissue in a mammal In a most preferred embodiment the mammal is a human.

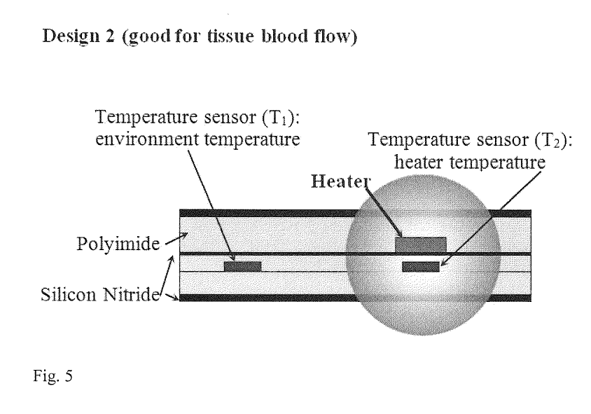

In one aspect of the invention, the flow sensor is as shown in FIG. 1. In one aspect of the invention, the flow sensor is as shown in FIG. 5. In one aspect of the invention, the flow sensor is as shown in FIG. 6. In one aspect of the invention, the flow sensor is as shown in FIG. 7. In one aspect of the invention, the flow sensor is as shown in FIG. 8.

Where a numerical range is provided herein for any parameter, it is understood that all numerical subsets of that numerical range, and all the individual integer values, and tenths thereof, contained therein, are provided, separately, as part of the invention. Thus, the range 0.8 mm to 2.0 cm includes the subset of distances such as 0.8-1.5 mm, the subset of distances which are 10-20 mm etc. as well as the distance 1.5 mm, the distance 2.0 mm, etc.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL RESULTS

### Results

When the temperature of an electrically-heated thermoresistive sensor increases, it loses thermal power to its surrounding until it reaches a thermal equilibrium in the presence of a moving fluid, which is defined by the following dynamic thermal energy balance equation:

$$\frac{{V}_{s}^{2}}{{R}_{s}} - ( a + b {υ}^{n} ) S ( {T}_{s} - {T}_{f} ) - mc \frac{d {T}_{s}}{d t} = 0 \qquad ( I )$$

where Vs is the voltage across the sensor, Rs is its resistance, Tₛ is the sensor temperature, T_{f} is the fluid temperature, m is the sensor mass, c is the specific heat, t is the time, ν is the fluid velocity and a, b and n are constants.

For thin film Au, there is a linear dependence between the sensor's active element resistance and its temperature:

Rₛ=R₀(1+α(Tₛ−T₀)) (II)

where α is the temperature coefficient of resistance (TCR) of developed Au film.

Thus equation (1) can be rewritten as:

$$\frac{{V}_{s}^{2}}{{R}_{s}} - ( a + b {υ}^{n} ) S ( {T}_{s} - {T}_{f} ) - \frac{mc}{α {R}_{0}} \frac{d {R}_{s}}{d t} = 0 \qquad ( III )$$

One of the most important sources of error in measuring flow rate is the change in the sensor calibration due to changes in the medium temperature. Periodic heating method requires only a single temperature point, where baseline conditions are established before the point of heating. So, the measurement is unaffected by spatial temperature gradients in comparison with continuous heating method (6). However, the flow measurements can still be disturbed if temperature variation occurs during the heating period. The temperature in physiologic systems is not constant, with excursions of up to 0.5-1° C. over periods of minutes. Such variations can potentially disturb the flow measurements, especially if it happens during the heating period. This effect can be compensated for by integrating a separate temperature sensor (SCT) (7) outside the region of “thermal influence” of the heated SCF to continuously monitor baseline temperature.

A schematic for an improved blood flow meter is shown in FIG. 1. In an embodiment, it comprises three components: a temperature sensor (T₁) which is located outside the “thermal influence” area for temperature compensation during the heating period; a temperature sensor (T₂) which is located under the heater under a, e.g. a polyimide interlayer for the measurement of heater temperature, and a heater which can heat the temperature sensor T₂, e.g., 2° C. above the environmental temperature.

Typical blood flow meter operation procedures are as follows: (i) the heater is fully cooled down; (ii) both the medium (environment) temperature (by T₁) and the targeted resistance to heat (T₂) 2° C. above the medium temperature are measured by applying a small current without self-heating; (iii) during the initial heating period, the T₁ peak output is sampled to determine the medium thermal conductivity for subsequent compensation; (iv) T₂ is heated 2° C. above the baseline temperature and the output therefrom is compensated for the baseline temperature shifts with T₁; and (v) the flow rate is derived with thermal conductivity compensation.

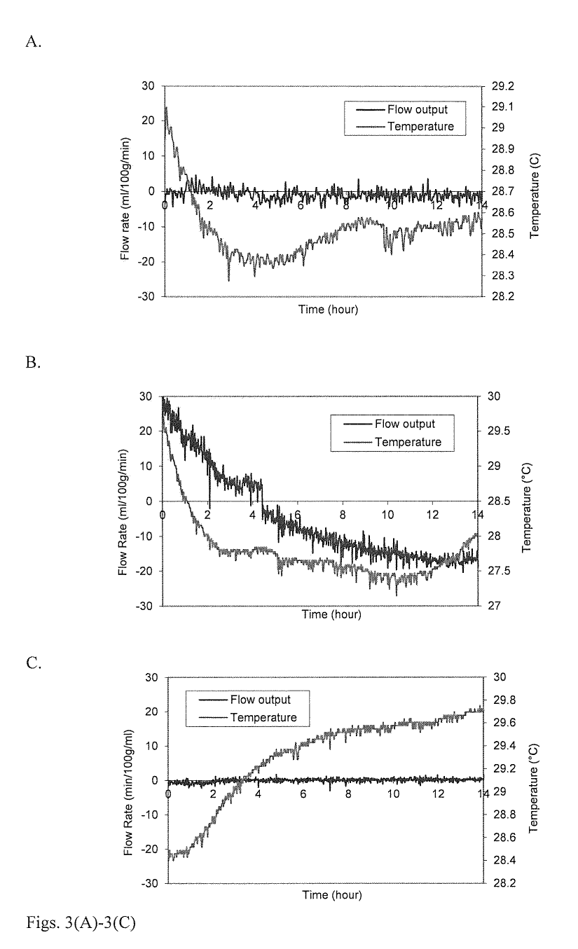

The approach presented here has several advantages than a previous approach in that it retains all the advantages of the previous approach and achieves at least four times higher accuracy. Examples of the superior accuracy and stability are shown in FIG. 3. FIG. 3 shows the long-term stability/accuracy test results from a previous (A) and two new devices, (B) and (C). Unpredictably, the new device where the heater is on the top and the temperature sensor is on the bottom shows much better accuracy than where the heater is on the bottom and the temperature sensor is on the top.

Polyimides (PI) have been used successfully as a substrate and insulation material for implants. However, high water absorption as well as high oxygen permeation of PI limit the performance of those microsensors using PI as a substrate. Herein, a permeability-reducing layer is preferably incorporated into the flexible substrate. In a preferred embodiment, thin silicon nitride film (e.g., 40 nm˜100 nm) is sputtered on a flexible PI substrate to reduce water or gas permeability and so improve sensor performance.

In an embodiment the heater element is a micro-heater comprising patterned Au or Pt film (e.g., 1200 nm thick, 20 μm wide). Temperature sensors as set forth in the designs in the figures T₁ and T₂ are, in embodiments, Au or Pt RTD (Resistance Temperature Detector) or thin film thermistor or thermocouple.

In summary, herein are disclosed blood flow meters offering many advantages, including unpredicted superior performance parameters, over prior sensors.

### REFERENCES

1. D. Mette, R. Strunk, M. Zuccarello, Translational stroke research 2, 152 (2011).
2. S. C. Lee, J. F. Chen, S. T. Lee, J Clin Neurosci 12, 520 (2005).
3. A. Dagal, A. M. Lam, Curr Opin Anesthesio 24, 131 (2011).
4. G. Rosenthal, R. O. Sanchez-Mejia, N. Phan, J. C. Hemphill, C. Martin, G. T. Manley, J Neurosurg 114, 62 (2011).
5. F. Verdii-López, J. M. Gonzalez-Darder, P. González-Lopez, and L. Botella Macia, Neurocirugia 21, 373 (2010).
6. C. Li, P. M. Wu, Z. Wu, C. H. Ahn, J. A. Hartings, R. K. Narayan, Proc. Of the 10th IEEE Sensors Conference (2011), Accepted.
7. C. Li, P. M. Wu, Z. Wu, C. H. Ahn, D. LeDoux, L. A. Shutter, J. A. Hartings, R. K. Narayan, Biomed Microdevices DOI: 10.1007/s10544-011-9589-4 (2011).

1. A blood flow meter for monitoring blood flow in a biological tissue comprising (a) a flow sensor comprising a first microelectrode, which flow sensor is capable of being heated by (b) a heater element positioned within 0.5 μm-100 μm of at least a portion of (a), and (c) a temperature sensor comprising a second microelectrode, wherein (a) and (c) are arranged on a flexible substrate and wherein (b) is positioned relative to (c) such that when (a) is heated by (b) to a stable target temperature of 0.5° C. to 3° C. above the temperature of the biological tissue in which the blood flow meter is situated, (c) is not within the thermal influence field generated by (b).

2. The blood flow meter of claim 1, wherein the flexible substrate comprises at least 5 (five) layers.

3. The blood flow meter of claim 1, wherein at least two layers of the flexible substrate comprise a flexible polymer.

4. (canceled)

5. The blood flow meter of claim 1, wherein at least two layers of the flexible substrate comprise silicon nitride.

6. The blood flow meter of claim 5, wherein an uppermost layer of the flexible substrate comprises silicon nitride and wherein a lowermost layer of the flexible substrate comprises silicon nitride.

7. The blood flow meter of claim 1, wherein the flexible substrate comprises at least 6 layers comprising, from top to bottom, with the bottom layer being exposed to the biological tissue or a blood vessel where blood flow is to be measured: a first silicon nitride layer adjacent to a first polyimide layer adjacent to a second silicon nitride layer adjacent to a second polyimide layer adjacent to a third polyimide layer adjacent to a third silicon nitride layer.

8-18. (canceled)

19. The blood flow meter of claim 12, having an air gap immediately adjacent to a polyimide layer underlying the interdigitated (a) and (b).

20. (canceled)

21. A blood flow meter for monitoring blood flow in a biological tissue, comprising (i) a flexible substrate; (ii) a flow sensor (x) comprising a first microelectrode, which flow sensor is capable of being heated by (iii) a heater element (y) positioned within 0.5 μm-100 μm of at least a portion of (x), wherein (x) is positioned on a first flexible substrate layer of a flexible substrate and (y) is positioned on a second flexible substrate layer of the flexible substrate, and wherein (x) and (y) are separated from each other by at least an intervening silicon nitride layer.

22-35. (canceled)

36. An array of two of the blood flow meters of claim 21, wherein the first blood flow meter and second blood flow meters are spatially separated on the same flexible substrate layers such that the first microelectrode of the first blood flow meter is outside the thermal influence field of the heater of the second blood flow meter and the first microelectrode of the second blood flow meter is outside the thermal influence field of the heater of the first blood flow meter.

37. The array of claim 36, having a configuration as shown in FIG. 7 or Design 4.

38. The blood flow meter of claim 1, wherein the flow sensor comprising a first microelectrode comprises a 4-wire configuration.

39. (canceled)

40. The blood flow meter of claim 1, wherein the temperature sensor quantitates the temperature of the medium in which the sensor for monitoring blood flow is situated, and wherein the sensor for monitoring blood flow is a component of an electrical circuit such that the output of the flow sensor is corrected for changes in temperature of the medium by the output of the temperature sensor.

41. The blood flow meter of claim 1, wherein the electrical circuit comprises an interface circuit.

42-66. (canceled)

67. A method for determining a blood flow in a tissue of subject comprising measuring the blood flow with the blood flow meter of claim 1 situated in the tissue of the subject.

68. The method of claim 67, comprising determining a baseline tissue temperature by causing a non-heating current to be applied to the sensor for monitoring blood flow and measuring the output of the sensor.

69. The method of claim 68, wherein the non-heating current is applied for between 0.1 and 20 seconds.

70. The method of claim 67, comprising effecting the temperature of the flow sensor to stably be 1° C. to 3° C. above the temperature of the tissue in which the blood flow meter is situated by causing a heating current to be applied to the sensor.

71-79. (canceled)

80. The method of claim 67, wherein the tissue is cerebral tissue.

81-83. (canceled)

84. A system for monitoring a blood flow in a tissue of a subject, comprising:
one or more data processing apparatus coupled to a blood flow meter of claim 1; and
a computer-readable medium coupled to the one or more data processing apparatus having instructions stored thereon which, when executed by the one or more data processing apparatus, cause the one or more data processing apparatus to perform a method of any of one of claims 67-83 so as to monitor the blood flow in a tissue of a subject.

85. A non-transitory computer-readable medium comprising instructions stored thereon which, when executed by a data processing apparatus, causes the data processing apparatus to perform a method of claim 67 so as to monitor blood flow in a subject.

86. (canceled)
